(19) United States
(12) Patent Application Publication
(10) Pub. No.: US 2015/0190349 A1
(43) Pub. Date: Jul. 9, 2015

(54) TRANSDERMAL THERAPEUTIC SYSTEM

(71) Applicant: Michael HORSTMANN, Neuwied (DE)

(72) Inventor: Michael HORSTMANN, Neuwied (DE)

(21) Appl. No.: 14/586,198

(22) Filed: Dec. 30, 2014

(30) Foreign Application Priority Data
Jan. 3, 2014 (DE) 10 2014 000 200.4

(51) Int. Cl. A61K 9/70; A61K 31/443; A61M 35/00

(52) U.S. Cl. CPC A61K 9/703; A61M 35/00; A61K 31/443

(57) ABSTRACT

Multilayer, active substance-containing transdermal therapeutic system (TTS) for releasing active substances via the skin to the human body.

A disadvantage of known transdermal therapeutic systems is that, due to insufficient cohesion, they are prone to smear during application on the skin and therefore do not reliably adhere to the skin.

To improve cohesion, a TTS (10) comprises two or more self-adhesive adhesive layers (1, 3, 3′, 3″) and at least one non-self-adhesive, high-tensile-strength and active substance-permeable intermediate layer (2, 2′, 2″); the said adhesive layers and intermediate layer(s) are alternately arranged and joined to one another.

The multilayer TTS is suited for the administration of pharmaceutical active substances via the skin for therapeutic purposes.

The invention relates to a multilayer active substance-containing patch (transdermal therapeutic system, TTS) for releasing active substances via the skin to the human body.

Under the designation of transdermal therapeutic systems (TTS), active substance-containing patches have already been introduced into the market in the pharmacotherapy of a number of diseases. The advantages of this form of active substance delivery are primarily the extended intervals of application, leading to improved patient compliance, as well as the avoidance of the first-pass effect (premature active substance metabolism in the case of oral administration), and the pharmacokinetically optimized plasma concentration-time profile, which ensures longer duration of action with fewer side effects.

For reason of the above advantages, TTS have been known for a number of years. Systems of this kind have been introduced into therapy for, for instance, estradiol, norethisterone acetate, nicotine, fentanyl, tulobuterol, rivastigmine, rotigotine, ethinyl estradiol/norelgestromin, buprenorphine or nitroglycerin and an increasing number of other active substances. The structure of transdermal therapeutic systems is typically thin and mostly two-layered, resulting, with the aid of the side directly facing the skin (adhesive layer), in an at least temporarily adhesive bond to the skin, via which the active substance (drug) is delivered.

TTS are, according to the state of the art, usually composed of a drug-impermeable carrier layer (also called backing or backing layer), an active substance-containing reservoir layer with a control membrane and adhesive layer, or of one or more matrix layers, if required with a pressure-sensitive adhesive layer for attachment to the skin, and a drug-impermeable protective layer (so-called release liner), which is intended to be removed prior to application.

To improve the permeation of active substance through the skin, use is made, in addition to polymers, resins and other pharmaceutical excipients, of system components which are liquid at room temperature and which serve, among other things, to adjust adhesion, to improve diffusion within the transdermal therapeutic system, and/or to improve the permeation of active substance via the skin.

Both active substances (drugs) and, primarily liquid, excipients may have the property of volatility and/or thermolability under process conditions, which is disruptive during manufacture. In order to avoid the problems caused by this, use may be made, during manufacture, of certain techniques known to those skilled in the art, more particularly, low-contamination forms of printing, of coating or of laminating with pure polymer/active substance mixtures, such as according to DE 4332094 A1 or DE 102006026060 A1.

Improving cohesion, particularly avoiding or preventing the “cold flow” of transdermal therapeutic systems has always been a predominant goal of development as systems that possess insufficient cohesion are prone to smear during application on the skin and thereby quickly become unsightly and do not reliably adhere to the skin. These problems are aggravated by the, generally, plasticizing effect of the active substance.

There have been many attempts in the past to improve the problem by adding consistency-improving substances in the case of silicone polymers (EP 0524776 A1), withdrawing moisture from the base polymer to make it more absorptive to active substances EP 2308480 A2), or by admixing hydrophilic polymers as additives to the adhesive polymer (WO 2005099676 A2). To date, none of these solutions has led to systems with drastically improved adhesion.

In addition, the use of frequently needed newly designed polymers has meanwhile become associated with high regulatory risks, which can in extreme cases result in the refusal of pharmaceutical approval and thereby exclusion from the market.

Multilayer transdermal therapeutic systems have been described in the prior art (e.g. EP 2298277 A1, U.S. Pat. No. 4,769,028 A), for example for achieving special release properties. However, the aforementioned, already known TTS likewise have the disadvantage of insufficient cohesion; in any case no special measures were provided to prevent cold flow. Insufficient cohesion makes the practical application of these TTS considerably more difficult.

The problem of cold flow could theoretically be alleviated also by reducing the thickness of the layer of adhesive because as the thickness of the flowable adhesive proportions decreases, the exit velocity at the margin becomes lower, on the basis of Stokes' Law. However, this measure is of very limited use as single adhesive layers of less than approximately 50 μm in thickness no longer adhere well to the skin; this applies, in any case, if no additional plasticizing in the rearward layer is carried out.

The object of the present invention is therefore to provide a transdermal therapeutic system having improved cohesion, wherein the so-called “cold flow” is prevented or at least reduced and which avoids the disadvantages of comparable systems known from the prior art. This object is solved by a multilayer transdermal therapeutic system according to the main claim, as well as by the embodiments indicated in the depending claims.

Accordingly, the present invention relates to a multilayer, active substance-containing transdermal therapeutic system (TTS) comprising a skin-side self-adhesive adhesive layer and an outer backing layer which is located opposite the said adhesive layer and which is impermeable to active substance. The TTS according to the present invention is characterized by having two or more self-adhesive adhesive layers and at least one non-self-adhesive intermediate layer which has high tensile strength and which is permeable to active substance, the said adhesive layers and intermediate layer(s) being alternately disposed and connected with one another, thereby forming a multilayer laminate.

The property of having high tensile strength means, in particular, that the tensile strength at 10% elongation is at least 0.1 N/mm², preferably at least 0.5 N/mm², more preferably at least 2 N/mm². The measuring method for determination of tensile strength will be explained further below.

Preferably, the non-self-adhesive intermediate layers (film layers) of the TTS have a tensile strength that corresponds to at least five times, particularly at least ten times, the tensile strength of the adhesive layer(s) (at 10% elongation). If the particular TTS has two or more adhesive layers that differ in terms of their tensile strength, the above indications (at least five times or at least ten times) refer to the adhesive layer having the highest tensile strength value.

The indications relating to the tensile strength of the non-self-adhesive layers and of the adhesive layers refer to the as-fabricated condition of the particular film or layer, that is, to the condition of the individual film or layer prior to assembly into a laminate or TTS.

Surprisingly, it has been found that the tensile strength of the whole system (TTS)—given a constant overall layer thickness—increases if the number of individual layers (adhesive layers and intermediate layers) that make up the system is raised (the individual layers should have a correspondingly smaller layer thickness in order to maintain the overall thickness of the system).

During the manufacture of the system described, single layers are initially applied, according to known methods, to prepared supporting films (carrier films), which are adhesively treated and are generally made of PET (polyethylene terephthalate), using the solvent-containing coating method employing doctor knife application, slot die application, spray application or roller application, in uniform layer thicknesses with approximately 10-80 (preferably 20-50) g/m² coating weight, and subsequently dried. The indications relating to layer thickness refer to the condition after drying.

In the case of two-sided, graded siliconization, direct winding-in-itself can be performed with the substrate. Laminating the system components, i.e. the individual layers of the TTS, one upon the other can be performed in any sequence desired, observing, however, the combination of adhesive layers and intermediate layers according to the present invention.

The surprising success according to the invention is a result of the combination of two or more adhesive layers, more particularly self-adhesive layers, which are separated by film-shaped intermediate layers, with active substance-diffusible (i.e. active substance-permeable) intermediate layers, which, however, impart mechanical stability (i.e. cohesion) and which are joined to their respective adjacent adhesive layers. Preferably, one intermediate layer is arranged between respective two adjacent adhesive layers, thereby forming a composite of layers.

Addition of the active substance, especially of a drug, to one or more layers is possible, both solvent-containing following intermediate drying processes, and solvent-free if the active substance is liquid at processing temperature or if it is added to a solvent that remains in the formulation. The active substance(s) or drug(s) may be contained in one or more adhesive layers, and/or in one or more intermediate layers. According to another embodiment, no active substance is added to the intermediate layer(s), so that in the as-fabricated condition the intermediate layer(s) is/are free of active substance(s).

The layers of adhesive and the intermediate layers of the TTS according to the invention can be manufactured in any sequence desired and laminated one upon the other in accordance with processes known to those skilled in the art. Optionally, a removable protective layer, which covers the skin-side adhesive layer and which is removed prior to use of the TTS, may be added.

As base polymers for the production of the self-adhesive layer(s) (layer(s) of adhesive), the following polymers are preferred: styrene-isoprene copolymers, polyisobutylene, silicones, acrylate copolymers, butyl rubber, polybutylene, styrene-isoprene, styrene copolymer, styrene-butadiene-styrene block copolymers. These base polymers may be used not only for the production of the skin-side adhesive layer but also for the production of the further self-adhesive adhesive layer(s).

Alkyl acrylate copolymers (preferably C₁-C₁₄ alkyl), in particular, and, more particularly, butyl methacrylate-methyl methacrylate copolymers are considered suitable as acrylate copolymers. As (co)monomers, the esters of acrylic acid or methacrylic acid, such as methyl acrylate, ethyl acrylate, propyl acrylate, methyl methacrylate, ethyl methacrylate, butyl acrylate, butyl methacrylate, isobornyl acrylate, isobornyl methacrylate, benzyl acrylate, benzyl methacrylate, lauryl acrylate, stearyl acrylate, isooctyl acrylate, 2-ethylhexyl acrylate, 2-ethylhexyl methacrylate, as well as acrylic acid and methacrylic acid are generally considered suitable.

The above list of base polymers is not intended to be limiting; basically all self-adhesive, physiologically acceptable polymers, singly or in combination (mixtures), are suitable.

Preferably, at least 40%-wt., preferably at least 50%-wt., more particularly at least 60%-wt., of the base material used for the production of the self-adhesive adhesive layers, preferably of all of the self-adhesive adhesive layers, is composed of the base polymers mentioned, preferably polyisobutylene, acrylic ester copolymers, silicone adhesive masses or mixtures of two or more of the aforementioned base polymers. The values given in %-wt. each relate to the dry weight.

Furthermore, the above-mentioned base material may contain one or more additives; additives that are suitable for pressure-sensitive adhesive layers are known to those skilled in the art. In particular, components such as resins (adhesive resins), oils, fillers and other pharmaceutically acceptable excipients can be used to advantage.

The thickness of the layers is generally not limited, but limitations may arise as a result of the methods of production employed. For example, the lower limit of the layer thickness may be determined by the precision of the methods of coating, and the upper limit may be determined by the remaining process economy of the drying process, which becomes more complex as the thickness increases. Hence, weights per unit area between 10 and 150 g/m² (preferably 20 to 50 g/m²; in each case in dried condition) can be rated as typical.

Preferably, the self-adhesive layers and/or the skin-side adhesive layer have an adhesion (peel force 90°) to steel of at least 1 N/cm², preferably at least 10 N/cm².

The adhesive layers contained in a TTS according to the invention may either have the same thickness or/and composition, or they may differ in terms of their thickness or/and composition. Furthermore, the active substance contents present in the individual adhesive layers of a TTS can be identical or different from one another.

The following polymers are preferred as the base polymers for producing the non-self-adhesive intermediate layer(s): methacrylate copolymers, high-molecular-weight polyisobutylene (preferably >150,000 g/mol, particularly >1,100,000 g/mol), polyvinyl alcohol, polyvinylpyrrolidone, styrene-isoprene copolymers, starch derivatives, pullulan, cellulose derivatives (e.g. carboxymethyl cellulose, ethyl cellulose, methyl cellulose, hydroxymethyl cellulose, hydroxypropyl methyl cellulose).

In particular, aminoalkyl methacrylate copolymers, preferably copolymers containing diethylaminoethyl residues, are also considered suitable as the said methacrylate copolymers.

The above listing of base polymers is not intended to be limiting; basically, all non-self-adhesive, physiologically acceptable polymers and mixtures thereof are suited.

With regard to the non-self-adhesive intermediate layers, it is furthermore preferred for these layers to possess an adhesion to steel (peel force 90°) of below 0.01 N/cm², preferably below 0.001 N/cm².

Preferably, at least 40%-wt., preferably at least 50%-wt., more particularly at least 60%-wt., of the base material for the production of the non-adhesive intermediate layers (2, 2′, 2″), preferably of all the intermediate layers, is composed of the base polymers mentioned, preferably polyvinylpyrrolidone, methacrylate copolymers, ethinylene-vinyl acetate, polyvinyl acetate, polyethylene, modified cellulose, modified starch, polyvinyl alcohol, or of mixtures of two or more of the aforementioned substances. The values given in %-wt. each relate to the dried weight.

Generally, the tensile strength of the non-self-adhesive intermediate layers can be improved by increasing the polymer proportion and/or a by correspondingly decreasing the filler proportion. It may therefore, for the purpose of improving tensile strength, be of advantage if the polymer proportion in the intermediate layers be increased to at least 70%-wt., preferably at least 80%-wt., particularly at least 90%-wt.

Furthermore, the non-self-adhesive intermediate layers may contain one or more additives; additives suited for this purpose are known to the skilled artisan. When choosing the kind and quantity of the additives, it should be ensured that the (intermediate) layers which are produced using these additives have a high tensile strength, more particularly that they have a tensile strength of more than 0.1 N/mm², preferably more than 2 N/mm² (at 10% elongation).

The thickness of the intermediate layers is generally not limited, but limitations may arise as a result of the methods of production employed. For example, the lower limit of the layer thickness may be determined by the precision of the methods of coating, and the upper limit may be determined by the remaining process economy of the drying process, which becomes more complex as the thickness increases. Hence, weights per unit area between 10 and 150 g/m² (preferably 20 to 50) can be rated as typical.

The intermediate layer(s) is/are active substance-diffusible (active substance-permeable) layers or films, that is, they are pervious to the active substance(s), especially drugs, contained in the TTS. Preferably, the intermediate layer(s), or at least one of the intermediate layers, are formed as a film or have the shape of a film.

The intermediate layers contained in a TTS according to the invention may either have identical thickness or/and composition, or they may differ in terms of their thickness or/and composition. Also, commercially available film materials may be chosen as the intermediate layers, provided they have the tensile strength demanded according to the invention (see above).

All of the layers of the TTS (both the adhesive layers and the intermediate layers) can be produced using conventional techniques of dissolving, mixing, coating and temperature-protected drying, but also by molding characterized only by heat. In the processes of the latter type, which are generally also referred to as extrusion, the mass is plasticated by kneaders and coatingly applied via a slot die.

The materials suitable for the production of the said active substance-impermeable backing layer (as well as of the removable protective layer) are known to those skilled in the art (e.g. films made of PET, polyethylene, polypropylene, PVC; metal foils). As a result of the presence of the backing layer, on the one hand a possible loss of active substance to the outside is prevented, and on the other hand the TTS is protected from potentially adhering to (the patient's) textiles.

The active substance proportion may amount to, preferably, 0.1 to 60%-wt., more particularly 1 to 30%-wt. or 5 to 25% wt., in those layers in which, according to the above description, such a proportion is intended to be present. At any rate, the proportions of the base polymer(s), of the additives (optional) and of the active substances (optional) contained in the respective base material or in the respective layer (adhesive layer or non-self-adhesive layer) add up to 100%-wt., relative to the dried state.

The addition of the active substances can be effected by adding active substance to all, individual, or else to only a single one of these layers. To this end, methods of production can be chosen in which active substance(s) and excipient/excipients (such as base polymers, additives, excipients) are jointly dissolved, suspended or dispersed, with subsequent coating and drying. For example, methods of production may be chosen in which the active substance (in particular, a drug) itself is used as a solvent (or suspending liquid or dispersing liquid) for the base polymer or base polymers and/or the additives.

In particular cases, for example where volatile active substances are used, it can be advantageous if at least one of the, preferably film-shaped, intermediate layers is made up of two or more plies of the same base polymers (and, if required, additives). In that case, the base polymers and, if required, additives, of the film-shaped intermediate layer are dissolved in a liquid active substance (more particularly, in a volatile active substance) and applied, as a second ply, onto a first ply which has been prepared beforehand (preferably of the same base material) and which is a pure ply, that is, a ply produced without active substance. The rapid equilibration of the active substance, which occurs subsequently, within the two plies, which are joined to one another, produces a uniform, active substance-containing intermediate layer.

In any case, because of the short diffusion paths, a spontaneous distribution of the active substance in the system components through equilibration, if desired, within hours up to days after fabrication, is easily possible.

The overall thickness of the multilayer systems (TTS) according to the invention is preferably 50 μm to 2 mm, more particularly 100 μm to 500 μm.

Tensile strength (in N/mm²) is determined in a manner known as such, by means of the tensile test (tension testing). To this end, test specimens (tensile specimens) are produced from the base material to be tested (films or layers), from the TTS layer to be tested, or from the TTS to be tested (entire system), for example by die-cutting. The test specimens used for determining tensile strength are not equipped with a carrier film or supporting film as the presence of such a film would distort the measurement result.

The specimens or blanks each have a width of 1 cm, with linear parallelly extending edges (in longitudinal direction). For better comparability of the measurements, the sample specimens should have approximately the same thickness; however, this is not strictly necessary as tensile force is proportional to the thickness of the specimens, and conversion is therefore easily possible.

The test specimen is clamped between the jaw clamps of a tensile testing machine, the overall gauge length of the test specimen being 5 cm. This length corresponds to the section of the test specimen which is located between the opposing jaw clamps.

Then, the test specimen is extended in the tensile testing machine at a constant rate of deformation (1% elongation per second), and the change in length of the test specimen is measured. In the process, the test specimen is subjected to integrated force measurement, with the mean value being determined over 5 mm elongation (=10% elongation). The tensile elongation value in N/mm² relates to the elongation measured at 10%.

The measurement is carried out at 25° C., 50% relative air humidity and an atmospheric pressure of 800 to 1060 hPa; the specimens are stored (equilibrated), prior to the measurement, for a period of at least 12 h under the conditions mentioned.

### EXAMPLES

### Example 1

### Preparation of a System (TTS) According to the Invention

a) 20 g polyisobutylene (e.g. Oppanol® 100, BASF; approx. 1,110,000 g/mol) is completely dissolved in 60 g n-heptane and coated, with a slot width of approx. 70 μm, onto a siliconized PET film (50 μm thickness), serving as a supporting or carrier film. After drying for 4 min at 60° C. and subsequently for 5 min at 80° C., a uniform self-adhesive layer of 17 g/m² is obtained.
b) 20 g of neutral polymethylmethacrylate (e.g. Plastoid® B, Evonik; butyl methacrylate-methyl methacrylate copolymer, approx. 150,000 g/mol) is completely dissolved in 60 g ethyl acetate and coated, with a slot width of approx. 120 μm, onto a siliconized PET film (50 μm). After drying for 20 min at 70° C., a uniform, non-self-adhesive layer (intermediate layer) of 30 g/m² is obtained on the substrate (PET film).
c) 20 g polyisobutylene (e.g. Oppanol® 100, BASF) is completely dissolved in 60 g n-heptane and, with a slot width of approx. 100 μm, coated onto a siliconized PET film (50 μm). After drying for 7 min at 60° C. and subsequently for 10 min at 80° C., a uniform self-adhesive layer of 25 g/m² is obtained.
d) 5 g of alkaline polymethylmethacrylate (e.g. Eudragit® E 100, Evonik; dimethylaminoethyl methacrylate-butyl methacrylate-methyl methacrylate copolymer, approx. 47,000 g/mol) is completely dissolved in 30 g Miglyol® (triglyceride mixture of fatty acids of medium chain length, essentially caprylic, capric and lauric acid) and 20 g rivastigmine base (drug/active substance).
e) 1 g polyisobutylene (e.g. Oppanol® B200, BASF; approx. 4,000,000 g/mol), 10 g polyisobutylene (e.g. Oppanol® B100, BASF), 2 g polyisobutylene (e.g. Oppanol® B10, BASF; approx. 40,000 g/mol) and 5 g paraffin oil are completely dissolved in 50 g n-heptane and coated, with a slot width of approx. 200 μm, onto siliconized PET film (100 μm). After drying for 20 min at 70° C. and subsequently for 15 min at 95° C., a uniform, self-adhesive layer of 50-52 g/m² is obtained.
f) A 20 μm thick PET film is laminated to the intermediate product from a), on the adhesive side, and the siliconized PET film (50 μm; supporting layer) is removed and discarded. The PET film (20 μm) forms the backing layer of the system (TTS). The resulting laminate is laminated with the adhesive side to the intermediate product from b), and the siliconized PET film (50 μm) is removed and discarded.

The non-self-adhesive side originating from the intermediate product b) (that is, the non-self-adhesive intermediate layer) of the laminate, which laminate has become increasingly thick, is laminated onto the intermediate product c) -self-adhesive layer of 25 g/m²; the siliconized PET film (50 μm) is removed and discarded.

The self-adhesive surface or adhesive layer originating from the intermediate product c) is coated with the active substance-containing solution from d) with 25 g/m² (thereby forming a non-adhesive, active substance-containing intermediate layer), and simultaneously or subsequently intermediate product e) is laminated thereto with the adhesive side (self-adhesive layer). The siliconized PET film (100 μm) forms the protective layer (release liner) of the multilayer TTS thus formed.

The sequence of the layers, joined to one another, of this TTS is as follows (from distal/outside to proximal/on the skin side): backing (PET film, 20 μm); adhesive layer (polyisobutylene; 17 g/m²); non-adhesive intermediate layer (neutral polymethylmethacrylate; self-adhesive layer (polyisobutylene, 25 g/m²); intermediate layer (alkaline polymethylmethacrylate); self-adhesive layer (polyisobutylene, 50-52 g/m²); protective layer (siliconized PET film, 100 μm).

After an equilibration time of around 24 hours, blanks are die-cut. The blanks can be adhered directly to the skin for therapeutic purposes after the protective layer (siliconized PET film, 100 μm) has been removed and discarded.

### Example 2

### Preparation of Another System (TTS) According to the Invention

a) 14 g polyisobutylene (e.g. Oppanol® B100, BASF) and 5 g polyisobutylene of a lower molecular weight (e.g. Oppanol® B10, BASF; approx. 40,000 g/mol) are completely dissolved in 60 g n-heptane and coated, with a slot width of approx. 100 μm, onto a siliconized PET film (50 μm thickness), which serves as a supporting or carrier film. After drying for 7 min at 60° C. and subsequently for 10 min at 80° C., a uniform self-adhesive layer of 25 g/m² is obtained.
b) 20 g of alkaline polymethylmethacrylate (e.g. Eudragit® E 100, Evonik; see hereinabove) is completely dissolved in 60 g ethyl acetate and coated, with a slot width of approx. 120 μm, onto a siliconized PET film (supporting film, 50 μm). After drying for 20 min at 70° C., a uniform, non-self-adhesive layer of 30 g/m² is obtained on the substrate.
c) 20 g polyisobutylene (e.g. Oppanol® 100, BASF) is completely dissolved in 60 g n-heptane and coated, with a slot width of approx. 100 μm, onto a siliconized PET film (50 μm). After drying for 7 min at 60° C. and subsequently for 10 min at 80° C., a uniform, self-adhesive layer of 25 g/m² is obtained.
d) 5 g alkaline polymethylmethacrylate (e.g. Eudragit® E 100, Evonik; see hereinabove) is completely dissolved in 30 g nicotine base.
e) 20 g polyisobutylene (e.g. Oppanol® 100, BASF) and 5 g paraffin oil are completely dissolved in 50 g n-heptane and coated, with a slot width of approx. 200 μm, on a siliconized PET film (100 μm). After drying for 20 min at 70° C. and subsequently for 15 min at 95° C., a uniform, self-adhesive layer of 50-52 g/m² is obtained.
f) A 20 μm thick PET film is laminated to the intermediate product from a), on the adhesive side, and the siliconized PET film (50 μm) is removed and discarded.

The resultant laminate is laminated with the adhesive side to the intermediate product from b), and the siliconized PET film (50 μm) is removed and discarded.

The non-self-adhesive side, originating from intermediate product b), of the laminate, which laminate has become increasingly thick, is laminated to intermediate product c), the siliconized PET film (50 μm) is removed and discarded. The self-adhesive surface is coated with the solution from d), with 30 g/m², and simultaneously intermediate product e) is laminated thereto with the adhesive side.

After at least 24 hours of equilibration time, blanks of 20 cm² are die-cut. The blanks can be directly adhered to the skin for therapeutic purposes after the siliconized PET film (100 μm) has been discarded and removed.

### Example 3

### Preparation of a Further System According to the Invention

a) 14 g polyisobutylene (e.g. Oppanol® B100, BASF) and 5 g polyisobutylene of a lower molecular weight (e.g. Oppanol® B10, BASF) are completely dissolved in 60 g n-heptane and coated, with a slot width of approx. 100 μm, onto an untreated PET film (20 μm). After drying for 7 min at 60° C. and subsequently for 10 min at 80° C., a uniform self-adhesive layer of 25 g/m² is obtained.
b) 20 g of alkaline polymethylmethacrylate (e.g. Eudragit® E 100, Evonik) is completely dissolved in 60 g ethyl acetate and coated, with a slot width of approx. 120 μm, onto a siliconized PET film (50 μm). After drying for 20 min at 70° C., a uniform, non-self-adhesive layer of 30 g/m² is obtained on the substrate.
c) 20 g polyisobutylene (e.g. Oppanol® 100, BASF) is completely dissolved in 60 g n-heptane and coated, with a slot width of approx. 100 μm, onto a siliconized PET film (100 μm). After drying for 7 min at 60° C. and subsequently for 10 min at 80° C., a uniform, self-adhesive layer of 25 g/m² is obtained.
d) 5 g alkaline polymethylmethacrylate (e.g. Eudragit® E 100, Evonik) is completely dissolved in 30 g nicotine base.
e) The intermediate product from a) is laminated, with the adhesive side, to the intermediate product from b), and the siliconized PET film (50 μm), the auxiliary support from b), is removed and discarded.

The solution from d) is coated, with 20 g/m², on the exposed, non-self-adhesive polymethylmethacrylate side of the resultant laminate, and intermediate product c) is subsequently laminated thereto with its adhesive side.

f) After an equilibration time of at least 24 hours, blanks of 20 cm² are die-cut. The blanks can be adhered directly to the skin for therapeutic purposes after the siliconized PET film (100 μm) has been removed and discarded.

### Example 4

### Preparation of a Further System According to the Invention

a) 14 g polyisobutylene (e.g. Oppanol® B100, BASF) and 5 g polyisobutylene of a lower molecular weight (e.g. Oppanol® B10, BASF) are completely dissolved in 60 g n-heptane and coated, with a slot width of approx. 100 μm, onto a siliconized (a less strongly separating type) PET film (100 μm). After drying for 7 min at 60° C. and subsequently for 10 min at 80° C., a uniform self-adhesive layer of 25 g/m² is obtained.
b) 20 g of alkaline polymethylmethacrylate (e.g. Eudragit® E 100, Evonik) is completely dissolved in 60 g ethyl acetate and coated, with a slot width of approx. 120 μm, onto a siliconized PET film (50 μm). After drying for 20 min at 70° C., a uniform, non-self-adhesive layer of 30 g/m² is obtained on the substrate.
c) 20 g polyisobutylene (e.g. Oppanol® 100, BASF) is completely dissolved in 60 g n-heptane and coated, with a slot width of approx. 100 μm, onto a siliconized PET film (50 μm). After drying for 7 min at 60° C. and subsequently for 10 min at 80° C., a uniform, self-adhesive layer of 25 g/m² is obtained.
d) The intermediate product from a) is laminated with its adhesive side onto the intermediate product from b), and the siliconized PET film (50 μm) is removed and discarded. The intermediate product is laminated with its adhesive side onto the adhesive side of the intermediate product from c), and, after removal of the siliconized PET film (50 μm), is held ready in this exposed condition.
e) 14 g polyisobutylene (e.g. Oppanol® B100, BASF) and 5 g polyisobutylene of lower molecular weight (e.g. Oppanol® B10, BASF) are completely dissolved in 60 g n-heptane and coated, with a slot width of approx. 100 μm, onto an untreated PET film (21 μm). After drying for 7 min at 60° C. and subsequently for 10 min at 80° C., a uniform self-adhesive layer of 25 g/m² is obtained.
f) 20 g of alkaline polymethylmethacrylate (e.g. Eudragit® E 100, Evonik) is completely dissolved in 60 g ethyl acetate and coated, with a slot width of approx. 120 μm, onto a siliconized PET film (50 μm). After drying for 20 min at 70° C., a uniform, non-self-adhesive layer of 30 g/m² is obtained on the substrate.
g) 20 g polyisobutylene (e.g. Oppanol® 100, BASF) is completely dissolved in 60 g n-heptane and coated, with a slot width of approx. 100 μm, onto a siliconized PET film (50 μm). After drying for 7 min at 60° C. and subsequently for 10 min at 80° C., a uniform, self-adhesive layer of 25 g/m² is obtained.
h) The intermediate product from e) is laminated with its adhesive side to the intermediate product from f), and the siliconized PET film (50 μm) is removed and discarded. The intermediate product is laminated, on the adhesive side, to the adhesive side of the intermediate product from g) and, after removal of the siliconized PET film (50 μm), is held ready in this exposed condition.
i) 5 g alkaline polymethylmethacrylate (e.g. Eudragit® E 100, Evonik) is completely dissolved in 30 g nicotine base.
j) The solution from i) is coated, with 25 g/m², onto the exposed self-adhesive surface from d), and subsequently intermediate product h) is laminated thereto with the adhesive side.

After an equilibration time of at least 24 hours, blanks of 20 cm² are die-cut. The blanks can be adhered directly to the skin for therapeutic purposes, after the siliconized PET film (100 μm) has been removed and discarded.

### Example 5

### Preparation of Another System According to the Invention

a) 20 g polyisobutylene (e.g. Oppanol® 100, BASF) is completely dissolved in 60 g n-heptane and coated, with a slot width of approx. 100 μm, onto a siliconized PET film (50 μm). After drying for 7 min at 60° C. and subsequently for 10 min at 80° C., a uniform self-adhesive layer of 25 g/m² is obtained.
b) 20 g polyvinylpyrrolidone (e.g. Kollidon® 90) and 30 g rotigotine are completely dissolved in 120 g ethanol and coated, with a slot width of approx. 120 μm, onto a siliconized PET film 50 μm. After drying for 10 min at 70° C., a uniform non-self-adhesive layer of 22 g/m² is obtained on the substrate.
c) 20 g polyisobutylene (e.g. Oppanol® 100, BASF) is completely dissolved in 60 g n-heptane and coated, with a slot width of approx. 100 μm, onto a siliconized PET film 50 μm. After drying for 7 min at 60° C. and subsequently for 10 min at 80° C., a uniform self-adhesive layer of 25 g/m² is obtained.
d) 20 g polyvinylpyrrolidone (e.g. Kollidon® 90) and 30 g rotigotine are completely dissolved in 120 g ethanol and coated, with a slot width of approx. 120 μm, onto a siliconized PET film (50 μm). After drying for 10 min at 70° C., a uniform self-adhesive layer of 22 g/m² is obtained on the substrate.
e) 20 g polyisobutylene (e.g. Oppanol® 100, BASF) and 5 g paraffin oil are completely dissolved in 50 g n-heptane and coated, with a slot width of approx. 200 μm, onto a siliconized PET film (100 μm). After drying for 20 min at 70° C. and subsequently for 15 min at 95° C., a uniform, self-adhesive layer of 50-52 g/m² is obtained.
f) A 20 μm thick PET film is laminated, with the adhesive side, to the intermediate product from a), and the siliconized PET film 50 μm is removed and discarded.

The resultant laminate is laminated with the adhesive side to the intermediate product from b), and the siliconized PET film 50 μm is removed and discarded.

The non-self-adhesive side, originating from intermediate product b), of the laminate, which laminate has become increasingly thick, is laminated to intermediate product c), the siliconized PET film (50 μm) is removed and discarded. The resultant laminate is laminated with its adhesive side to the intermediate product from d), and the siliconized PET film (50 μm) is removed and discarded. Subsequently, intermediate product e) is laminated, with its adhesive side, to the surface.

After around 1 h of equilibration time, blanks of 30 cm² are die-cut. The blanks can be directly adhered to the skin for therapeutic purposes after the siliconized PET film (100 μm) has been removed and discarded.

### Example 6

### a) Preparation of a Partial Laminate According to the invention

A non-adhesive layer with a layer thickness of 10 μm is produced from a solution (30%-wt.) of Plastoid® B in ethyl acetate. Plastoid® B (Evonik Industries AG) is a copolymer of butyl methacrylate and methyl methacrylate with an average mass average molar mass of 150,000 g/mol.

Furthermore, a self-adhesive layer with a layer thickness of 20 μm is prepared in accordance with Example 5e) hereinabove.

The upper and lower sides of this self-adhesive layer are each covered with a non-self-adhesive layer of Plastoid® B (see above) and assembled into a three-layered laminate. Subsequently, five additional layers (non-self-adhesive and adhesive layers, respectively) are combined with the three-layered laminate. The sequence of the layers is such that each non-self-adhesive (intermediate) layer is followed by an adhesive layer, and vice versa.

From the laminate thus obtained, which has an overall layer thickness of approx. 50 μm, test specimens for tensile strength measurements are obtained by die-cutting, as described further above.

### b) Preparation of a Comparative Example

A three-layered laminate serves as a comparative example and is prepared as follows:

From a solution (30%-wt.) of Plastoid® B in ethyl acetate, a non-self-adhesive layer having a layer thickness of 25 μm is produced. Furthermore, a self-adhesive layer according to Example 5e) is prepared, having a layer thickness of 20 μm. The upper and lower sides of this self-adhesive layer are each covered with a non-self-adhesive layer of Plastoid® B (25 μm, see above) and assembled into a three-layered laminate.

From the three-layered laminate thus obtained, having an overall layer thickness of approx. 50 μm, test specimens for tensile strength measurements are obtained by die-cutting, as described further above.

### c) Tensile Strength Measurements

The test specimens prepared from the inventive partial laminate and the three-layered comparative laminate were subjected to a tensile test (as described in detail further above). Given approximately identical thickness of the total laminate, the tensile strength values achieved with the partial laminates according to the invention were, on average, twice as high as the tensile strength values of the comparative examples.

### DRAWINGS

The appended drawings serve merely to illustrate the invention, without any intention to limit the scope of protection. The shapes and proportions shown are not true to scale.

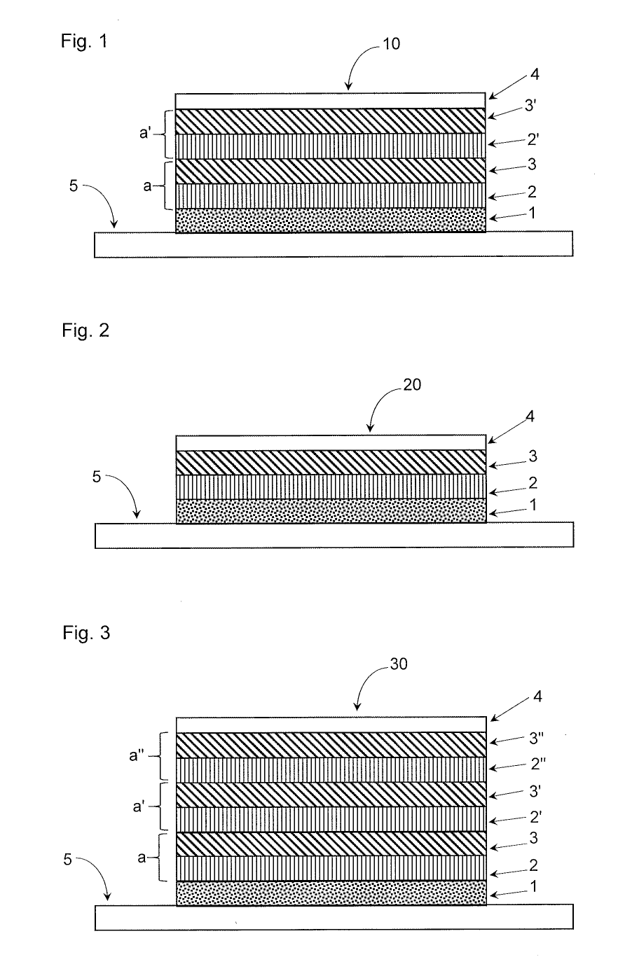

FIG. 1 shows—in sectional representation—an embodiment of a multilayer transdermal therapeutic system (10) according to the present invention, in accordance with claim 1. In the embodiment shown here, there is present a repeatedly arranged double layer (a, a″), each one composed of a self-adhesive layer (3, 3′) and a non-self-adhesive intermediate layer (2, 2′).
(1) skin-side adhesive layer (self-adhesive)
(2), (2′) non-self-adhesive, high-tensile-strength and active substance-diffusible layer
(3), (3′) (a further) self-adhesive layer (adhesive layer)
(a), (a′) once-repeated double layer, each one composed of a non-self-adhesive, high-tensile-strength and active substance diffusible (that is, active substance-permeable) intermediate layer (2) and (2′), respectively, and a self-adhesive layer (3) and (3′), respectively.
(4) active substance-impermeable backing layer (at the outside, distal)
(5) separating film (release liner), on skin-side adhesive layer (1).

FIG. 2 shows another embodiment of a multilayer transdermal therapeutic system (20) according to claim 1, but without repetition of a double layer.
(1) self-adhesive, skin-side layer
(2) non-self-adhesive, high-tensile-strength and active substance-diffusible layer (film-shaped intermediate layer)
(3) (another) self-adhesive layer
(4) active substance-impermeable backing layer
(5) separating layer (release liner), on the skin-side adhesive layer (1).

FIG. 3 shows another embodiment of a multilayer transdermal therapeutic system according to the present invention, in accordance with claim 1, with a twice-repeated double layer which consists of adhesive layer and intermediate layer.
(1) self-adhesive, skin-side layer
(2) non-self-adhesive, high-tensile-strength and active substance-diffusible layer (intermediate layer, film-shaped)
(3) (another) self-adhesive layer (adhesive layer)
(2), (2′), (2″) non-self-adhesive, high-tensile-strength and active substance-diffusible layer (film)
(3), (3′), (3″) (a further) self-adhesive layer (adhesive layer)
(a), (a′), (a″): twice-repeated double layer, each one consisting of a non-self-adhesive, high-tensile-strength and active substance-diffusible intermediate layer (2′, 2′, 2″) and a self-adhesive layer (3, 3′, 3″). In total, three double-layers (a, a′, a″) are arranged so as to be adjacent and joined to one another.
(4) active substance-impermeable backing layer (at the outside/distal)
(5) separating film (release liner), on skin-side adhesive layer (1).

1. Multilayer, active substance-containing transdermal therapeutic system comprising a skin-side self-adhesive adhesive layer (1) and an active substance-impermeable, outer backing layer (4) located opposite the said adhesive layer, wherein it comprises two or more self-adhesive adhesive layers (1, 3, 3′, 3″) and at least one non-self-adhesive, high-tensile-strength and active substance-permeable intermediate layer (2, 2′, 2″), the said adhesive layers and intermediate layer(s) being alternately arranged and joined to one another.

2. Transdermal therapeutic system according to claim 1, wherein the intermediate layer(s) (2, 2′, 2″), or at least one of the intermediate layers, is/are formed as (a) film(s).

3. Transdermal therapeutic system according to claim 1, wherein it comprises at least one double layer (a, a′, a″) which comprises a non-self-adhesive, high-tensile-strength and active substance-diffusible intermediate layer (2, 2′, 2″) and a self-adhesive layer (3, 3′, 3″) joined thereto.

4. Transdermal therapeutic system according to claim 3, wherein it comprises at least two, preferably two or three, of the said double layers (a, a′, a″) in successive arrangement, wherein a respective two adjacent double layers are joined to each other by means of a self-adhesive layer (3, 3′, 3″).

5. Transdermal therapeutic system according to claim 1, wherein the self-adhesive adhesive layers (1, 3, 3′, 3″), preferably all of the self-adhesive adhesive layers (1, 3, 3′, 3″), consist of at least 40%-wt., preferably at least 50%-wt., more particularly at least 60%-wt., of polyisobutylene, acrylic ester copolymers, silicone adhesive masses, or of mixtures of two or more of the aforementioned substances.

6. Transdermal therapeutic system according to claim 1, wherein the non-self-adhesive intermediate layers (2, 2′, 2″), preferably all of the intermediate layers, consist of at least 40%-wt., preferably at least 50%-wt., more particularly at least 60%-wt., polyvinylpyrrolidone, methacrylate copolymers, ethinylene vinyl acetate, polyvinyl acetate, polyethylene, modified cellulose, modified starch, polyvinyl alcohol, or of mixtures of two or more of the aforementioned substances.

7. Transdermal therapeutic system according to claim 1, wherein the tensile strength of the intermediate layer(s) (2, 2′, 2″) is at least 0.1 N/m², more particularly at least 0.5 N/mm², and especially preferably at least 2 N/mm².

8. Transdermal therapeutic system according to claim 1, wherein the tensile strength of the intermediate layer(s) (2, 2′, 2″) is at least five times, more particularly at least ten times, the tensile strength of the adhesive layer(s) (at 10% elongation).

9. Transdermal therapeutic system according to claim 1, wherein the intermediate layer(s) (2, 2′, 2″) have an adhesion to steel (peel force 90°) of below 0.01 N/cm², preferably below 0.001 N/cm².

10. Transdermal therapeutic system according to claim 1, wherein the self-adhesive layers (3, 3′, 3″) as well as the skin-side adhesive layer (layer 1) have an adhesion (peel force 90°) to steel of at least 1 N/cm², preferably at least 10 N/cm².

11. Transdermal therapeutic system according to claim 1, wherein the skin-side adhesive layer (1) is protected, prior to application, by a removable separating film (release liner), in order to protect its surface.

12. Transdermal therapeutic system according to claim 1, wherein at least one of the intermediate layers (2, 2′, 2″), preferably all intermediate layers, contain polyvinylpyrrolidone, the proportion of polyvinylpyrrolidone preferably amounting to at least 10%-wt., more particularly at least 20%-wt., especially preferably at least 30%-wt.

13. Transdermal therapeutic system according to claim 1, wherein the adhesive layers (3, 3′, 3″), preferably all of the adhesive layers (3, 3′, 3″) consist predominantly (i.e. at least 50%-wt., more particularly at least 60%-wt.) of polyisobutylene, acrylic acid copolymers or silicone polymers.

14. Transdermal therapeutic system according to claim 1, wherein the adhesive layers (3, 3′, 3″) consist predominantly (i.e. at least 50%-wt., more particularly at least 60%-wt.) of polyisobutylene or silicone polymers, and that the intermediate layers (2, 2′, 2″) contain polyvinylpyrrolidone as well as one or more active substances the solubility of which in polyvinylpyrrolidone is at least twice as high as the solubility in a 1:1 (w/w) mixture of polyvinylpyrrolidone and water.

15. Transdermal therapeutic system according to claim 1, wherein it contains rotigotine as an active substance.

16. Transdermal therapeutic system according to claim 1, wherein it comprises a self-adhesive skin-side layer (1), two double layers (a, a′) which are arranged on top of each other and are each composed of a high-tensile-strength, active substance-diffusible intermediate layer (2, 2′) and a self-adhesive layer (3, 3′), and an essentially active substance-impermeable backing layer (4), it being preferred that the self-adhesive layers (3, 3′) consist predominantly (i.e. at least 50%-wt., more particularly at least 60%-wt.) of polyisobutylene, and that the intermediate layers (2, 2′) contain methacrylate polymers.

17. Transdermal therapeutic system according to claim 1, wherein it comprises a self-adhesive skin-side layer (1), three double layers (a, a′, a″) which are arranged on top of each other and are each composed of a high-tensile-strength, active substance-diffusible intermediate layer (2, 2′, 2″) and a self-adhesive layer (3, 3′, 3″), and an essentially active substance-impermeable backing layer (4), it being preferred that the self-adhesive layers (3, 3′, 3″) consist predominantly (i.e. at least 50%-wt., more particularly at least 60%-wt.) of polyisobutylene, and that the intermediate layers (2, 2′, 2″) contain methacrylate polymers.

18. Transdermal therapeutic system according to claim 1, wherein it contains, as the active substance(s), a plasticizing, lipophilic active substance which, at 20° C., has a solubility of at least 0.1% (w/w) in ethanol, or a mixture of such active substances.

19. Transdermal therapeutic system according to claim 1, wherein it contains nicotine base as an active substance.
